(19) United States
(12) Patent Application Publication
(10) Pub. No.: US 2015/0238739 A1
(43) Pub. Date: Aug. 27, 2015

(54) SYSTEMS FOR THE REDUCTION OF LEAKAGE AROUND MEDICAL DEVICES AT A TREATMENT SITE

(71) Applicant: W. L. Gore & Associates, Inc., Newark, DE (US)

(72) Inventors: Edward H. Cully, Flagstaff, AZ (US); Jeffrey B. Duncan, Flagstaff, AZ (US); Kaylan M. Luber, Phoenix, AZ (US); William D. Montgomery, Flagstaff, AZ (US); Edward E. Shaw, Flagstaff, AZ (US)

(21) Appl. No.: 14/710,188

(22) Filed: May 12, 2015

Related U.S. Application Data
(63) Continuation of application No. 13/571,287, filed on Aug. 9, 2012.
(60) Provisional application No. 61/523,225, filed on Aug. 12, 2011.

(51) Int. Cl. A61M 29/02

(52) U.S. Cl. CPC A61M 29/02

(57) ABSTRACT

A flow reduction system is provided which includes any suitable system installable through and within the vasculature, configured to reduce flow of blood and other bodily fluids, and includes one or more components configured to fill spaces or “gutters” around and/or between medical devices installed in the vasculature.

### BACKGROUND

1. Field

The present disclosure relates to systems for reducing unwanted flow or leakage around medical devices installed in a treatment region, and more specifically for filling voids where medical devices are installed in a body lumen.

2. Discussion of the Related Art

Treatment of various portions of the vasculature can require the installation of one or more medical devices. A medical device can be any device or structure configured to provide and/or support a therapeutic use in the vasculature. For example, stents or stent grafts, valves, bifurcated stents, and drug-delivering devices can be implanted in the vasculature at a treatment region. Typical medical devices can have geometries that do not conform to the vasculature. Moreover, during a medical procedure, a plurality of medical devices can be installed in a single region of the vasculature in what are sometimes referred to as “chimney,” “snorkel,” or “sandwich” arrangements, for example, as shown and generally indicated at “d1” and “d2” in FIGS. 1A and 1B.

Sizes and shapes of anatomy, as well as pathologies involved vary greatly from patient to patient. Sizes, shapes and number of endovascular devices used (even within one procedure) vary greatly. Operator techniques for deploying the devices can also vary greatly. Thus, the cross section defined by the medical device(s) when installed in the vasculature may not equally correlate to the entire cross section of the vasculature where the medical device(s) is/are installed, thereby resulting in gaps or “gutters” or “gutter regions” with widely varied cross-sections and peripheries, as shown illustratively and indicated at “a” and “b” in FIGS. 1A and 1B.

Flow into and/or through the gutters can be unwanted. For instance, unwanted flow can pressurize an aneurysm or create other problems with the vasculature in the treatment region due to persistent leaking or perfusion.

Thus, a need exists to reduce unwanted flow or leakage into or through gutters. Those skilled in the art will recognize numerous advantages of disclosed embodiments over the prior art, including, for example, substantially reducing such flow in the gutter region with an implantable leakage reduction system.

### BRIEF DESCRIPTION OF THE DRAWINGS

In the Figures:

FIGS. 1A-1B illustrate cross sectional views of a section of human vasculature showing gutter regions which can result from deployment of two or more adjacent medical devices at a treatment site;

FIG. 2 illustrate an exemplary leakage reduction system comprising a frame with cover;

FIGS. 3A-3B illustrate an exemplary leakage reduction system comprising a filler;

FIG. 4 illustrates an exemplary leakage reduction system comprising a balloon;

FIG. 5 illustrates an exemplary leakage reduction system comprising a brush and a balloon;

FIGS. 6A-6E illustrate an exemplary leakage reduction system comprising a brush;

FIGS. 7A-7D illustrate an exemplary leakage reduction system comprising a brush and an occluder;

FIGS. 8A-8C illustrate an exemplary leakage reduction system comprising a laminated brush;

FIGS. 9A-9B illustrate an exemplary leakage reduction system comprising a brush and foam; and

FIGS. 10A-10C illustrate an exemplary leakage reduction system comprising a bag and brush.

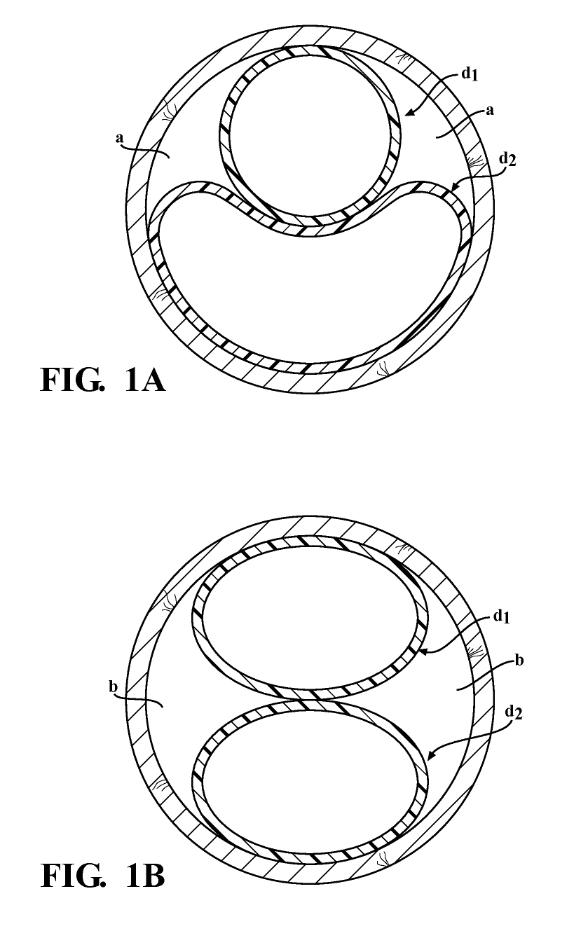

### DETAILED DESCRIPTION

The detailed description of various embodiments herein makes reference to the accompanying drawing figures, which show various embodiments and implementations thereof by way of illustration and best mode, and not of limitation. While these embodiments are described in sufficient detail to enable those skilled in the art to practice the embodiments, it should be understood that other embodiments can be realized and that mechanical and other changes can be made without departing from the spirit and scope of the present disclosure. Furthermore, any reference to singular includes plural embodiments, and any reference to more than one component can include a singular embodiment. Moreover, recitation of multiple embodiments having stated features is not intended to exclude other embodiments having additional features or other embodiments incorporating different combinations of the stated features.

As used herein, the term distal is used to denote the end of an exemplary device nearest to the treatment region within a patient's body. The term proximal is used to denote the end of an exemplary device nearest to the user or operator of the device.

As used herein, “leakage” means the unwanted or undesirable flow into or through a treatment region, where the flow is outside the lumen(s) or body(ies) defined by the medical device(s).

The present disclosure relates to a number of non-limiting, exemplary embodiments, each of which can be used alone or in coordination with one another. A leakage reduction system can be any suitable system installable within the vasculature and configured to reduce leakage of blood and other bodily fluids during and/or after a medical procedure. In various embodiments, the leakage reduction system can comprise one or more components configured to fill open spaces or “gutters” around medical devices installed in the vasculature. In another embodiment, the leakage reduction system can comprise one or more components with geometries configured to cover substantially all of the cross-sectional area of a vessel. In either of these embodiments, the leakage reduction system encourages flow through a lumen defined by installed medical devices and/or reduces the flow of blood and/or bodily fluids around the medical devices.

In various embodiments, the leakage reduction system can comprise any suitable structure configured to fill a gap. In these embodiments, the leakage reduction system comprises a support structure, including for example, a frame, gel, beads, alginate, a balloon, a brush, or any other suitable support structure. In general, any structure that provides volume and/or increased surface area (e.g., for coagulation) to a gap can be used.

The leakage reduction system can also comprise a coating or secondary structure, including, for example, a bag, an occluder disk, a balloon, foams, gels, and the like. Exemplary coatings can attach and/or laminate to the support structure, and/or the medical device itself, to further reduce leakage into or through a gutter.

The leakage reduction system can also comprise a radiopaque marker and a deployment mechanism. The radiopaque marker can allow a user to observe the position of the leakage reduction system in the body to properly position the system in a gutter. The deployment mechanism can be any suitable structure configured to removably attach the support structure or secondary structure to a delivery device. For example, the deployment mechanism can include a collar, threads, a clip, noose, snare, or any other suitable structure.

In various embodiments, the leakage reduction system can deploy endoluminally through any suitable medical device delivery system. The medical device delivery system can comprise a deployment mechanism, catheter, guidewire, or other suitable conduit for delivering the leakage reduction system to a treatment region. In these embodiments, the catheter, guidewire, or conduit can comprise a lumen configured to receive inputs and/or materials from the proximal end of the medical device delivery system and conduct the inputs and/or materials to the leakage reduction system at the treatment region.

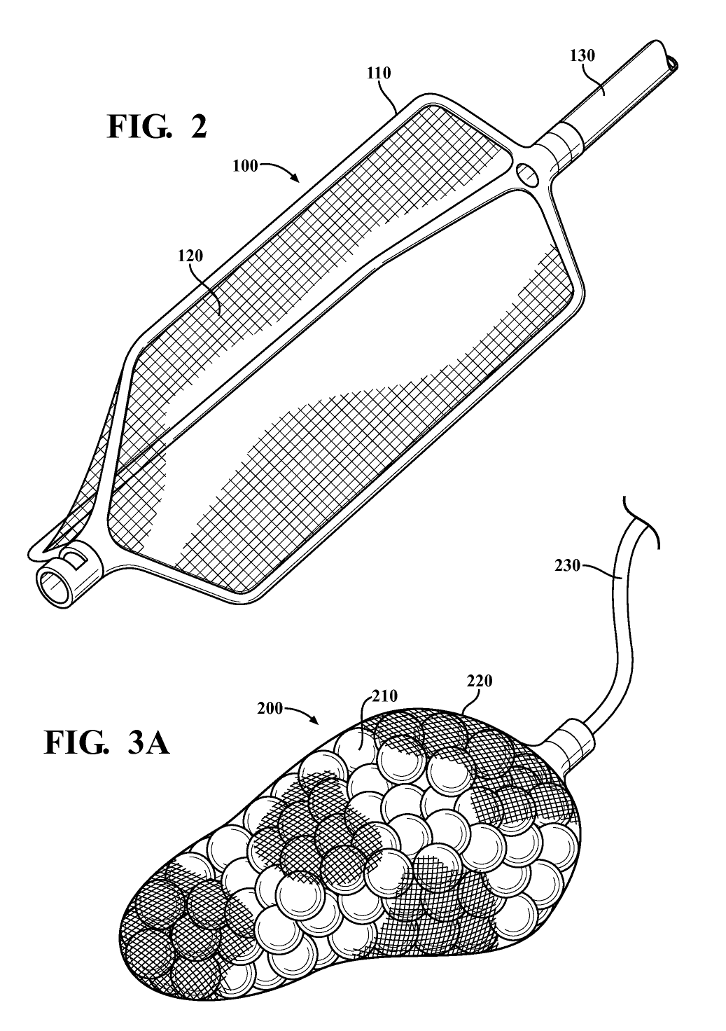

In various embodiments, and with reference to FIG. 2, leakage reduction system 100 can comprise a frame 110, a bag 120, and a deployment mechanism 130. Frame 110 can be of any suitable size and shape to fill a gutter. Frame 110 can support and couple to bag 120. Frame 110 can also comprise or otherwise couple to deployment mechanism 130. Frame 110 can be formed of laser cut tube, nitinol wire, or any other suitable biocompatible material. Bag 120 can be formed from ePTFE or any other suitable biocompatible material.

Bag 120 can couple to frame 110 such that frame 110 defines an open side. The open side can be installable around the medical device structure or vascular structure defining the gutter. Leakage reduction system 100 can also be oriented in the gutter to allow blood and/or bodily fluids to flow into the open side. When coupled to frame 110, bag 120 can be configured as an occluder bag. In various embodiments, the bag 120 can be porous so as to allow some blood and/or bodily fluid to flow therethrough when installed in the gutter. Over time, this flow can be substantially limited due to reduced porosity of bag 120 as a result of clotting, tissue in-growth, cross-linking, etc.

In use, the leakage reduction system 100 can be delivered endoluminally to a treatment region through any suitable medical device delivery system. Such a delivery system can include, for example, a catheter or other suitable sheath, after two or more medical device have been installed, where the installation has created a gutter. The delivery system can also include a deployment mechanism that releasably compresses the leakage reduction system 100 to a compressed size or state suitable for endoluminal delivery of the system to the treatment region. The system can then be expanded by releasing the deployment mechanism at the treatment region within the gutter. Deployment mechanisms can include constraining sleeves, sheaths, lines or tethers, or other structures suitable for releasably compressing the system to a size suitable for endoluminal delivery to the treatment region. This process can be repeated for installations with multiple gutters.

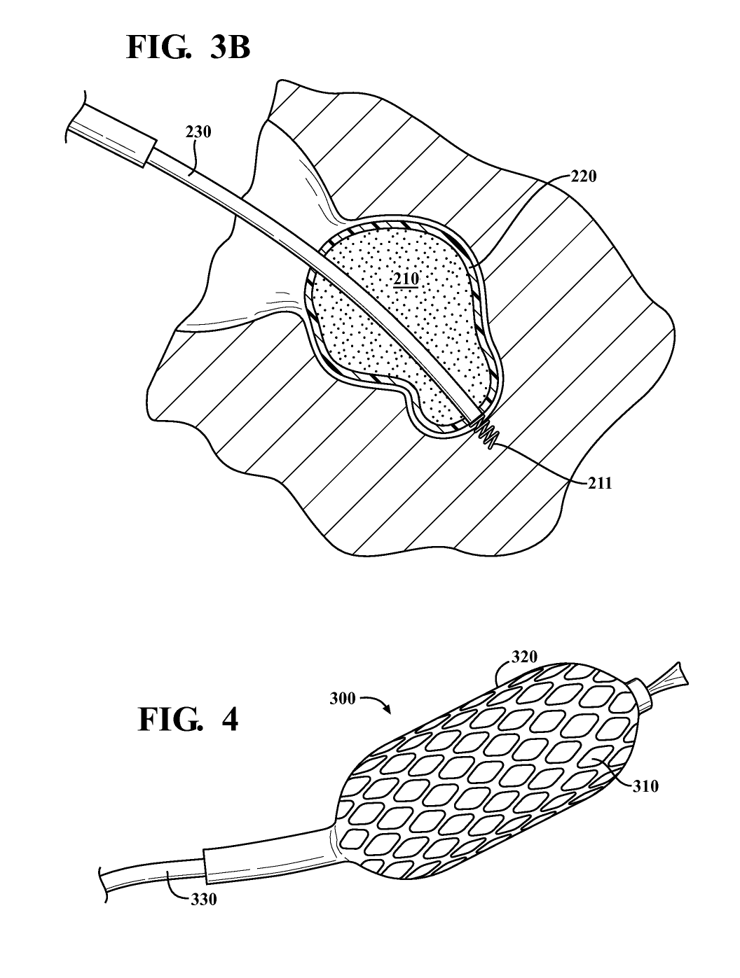

In various embodiments, and with reference to FIGS. 3A-3B, leakage reduction system 200 can comprise a filler 210, a bag 220, and a deployment mechanism 230. Bag 220 can contain and/or house filler 210 and couple to and/or define deployment mechanism 230. Filler 210 can be any suitable substance, including, for example, a gel, a foam, beads, alginate, a multi-part substance, and/or the like. Bag 220 can be made of any suitable material, such as, for example, ePTFE or any other suitable material.

In other embodiments, the leakage reduction system 200 can also comprise an attachment mechanism 211, as best shown in FIG. 3B. The attachment mechanism can be any suitable structure configured to couple and/or engage the vasculature. For example, attachment mechanism 211 can include a coil, a hook, a barb, an anchor, or any other suitable structure capable of coupling to and/or engaging a vascular wall or other tissue.

In a number of embodiments, filler 210 is a gel, foam, or multi-part material. In this embodiment, bag 220 can deploy endoluminally through a medical device delivery system on a catheter to a treatment region. Bag 220 can be empty or can comprise an initial part of a multi-part substance. Bag 220 can be positioned in the gutter and filled. Attachment mechanism 211 can further couple bag 220 to a vascular wall to stabilize bag 220 in the gutter. As noted above, the deployment can have a lumen configured to receive an input. In this embodiment, the input can be one or more of gel, foam, or a remaining part of multi-part substance, such as, for example, a swelling and/or a hardening or cross-linking compound. In response to the filling, bag 220 can fill the gutter and contact the medical devices and/or body lumen defining the gutter substantially reducing the flow of blood and/or bodily fluids through the gutter.

In another embodiment, filler 210 comprises beads. During insertion, bag 220 can be filled with beads or can be empty. Where bag 220 is filled with beads, it can be compressed by a delivery sheath and deploy endoluminally through a medical device delivery system on a catheter to a treatment region. Bag 220 can be positioned in the gutter. The delivery sheath can be removed or retracted to allow bag 220 to expand and fill the gutter. Alternatively, bag 220 can operatively couple to a lumen of a hollow catheter, in which case the beads can be fed through the catheter lumen to fill bag 220. As bag 220 fills, leakage system 200 obstructs the gutter and reduces the leakage of blood and/or bodily fluids around the medical devices at the treatment region.

In various embodiments, for example, with reference to FIG. 4, leakage reduction system 300 can comprise an inflatable member 320 and a deployment mechanism 330. Inflatable member 320 can be a balloon, a bladder, a bag or any other suitable structure that is configured to inflate under hydraulic or pneumatic pressure. Inflatable member can comprise texture 310. Texture 310 can be formed on the surface of inflatable member 320. Texture 310 can also be a structural component installed within inflation chamber defined by inflatable member 320 and protrude through the walls of inflatable member 320. Texture 310 can be configured to increase the friction exerted on a surface, when the surface is contacted by inflatable member 320.

Inflatable member 320 can deploy endoluminally through a medical device delivery system in a compressed or deflated configuration. Inflatable member 320 has an inflation cavity operatively couple to a catheter, such that the inflation cavity is in fluid communication with a lumen of a catheter. Upon reaching the treatment site, inflatable member 320 can be positioned in the gutter. In the deflated configuration, inflatable member 320 can receive fluid, such as, for example, water, saline, or other suitable fluid, through the catheter lumen. This fluid causes inflatable member 320 to expand outwardly from the deflated configuration. The expansion can cause inflatable member 320 and associated texture 310 to fill the gutter and engage the medical devices and/or vascular wall defining the gutter. Resulting friction created by the engagement of texture 310 can further retain and stabilize inflatable member 320 in the gutter.

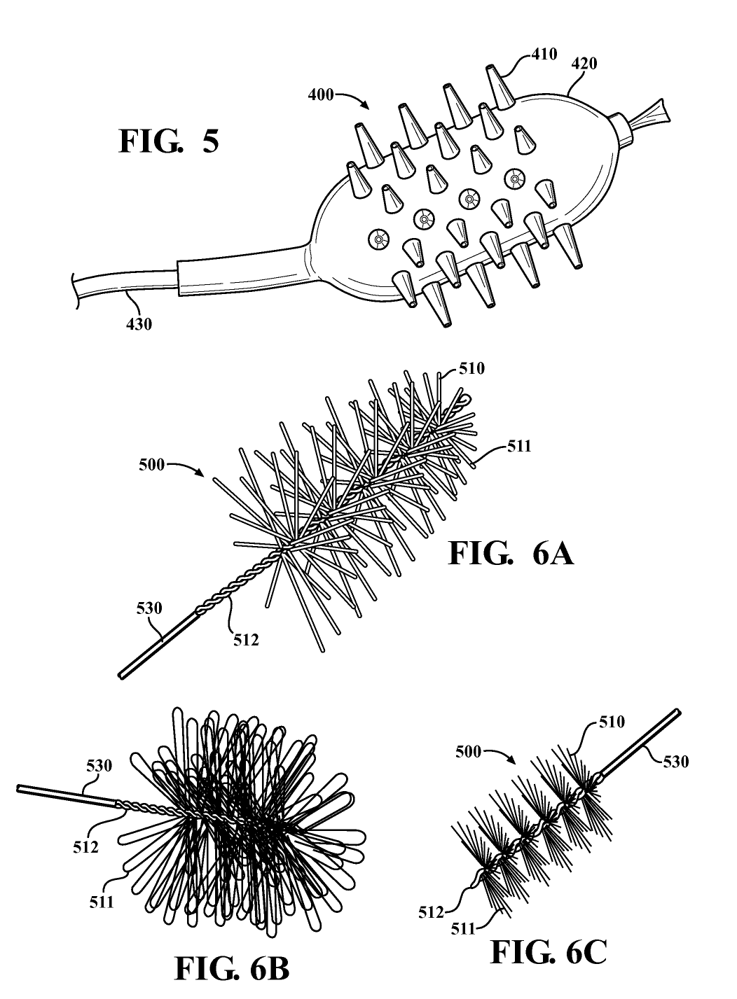

In various embodiments, for example, with reference to FIG. 5, leakage reduction system 400 can comprise a brush 410, an inflatable member 420 and a deployment mechanism 430. Brush 410 can comprise a support column coupled to a plurality a spaced bristles. Inflatable member 420 can be a balloon, a bladder, a bag or any other suitable structure configured to inflate under hydraulic or pneumatic pressure. Brush 410 can be installed within an inflation chamber defined by inflatable member 420, such that the bristles of brush 410 are covered by and elevate portions of the wall of inflatable member 420. Brush 410 can be configured to increase the friction exerted on a surface, when the surface contacts the elevated portions of inflatable member 420.

Inflatable member 420 can deploy endoluminally through a medical device delivery system in a compressed or deflated configuration. The bristles of brush 410 can be sufficiently soft and/or flexible that inflatable member 420 compresses the bristles during deployment through the catheter. The assembly of brush 410 and inflatable member 420 can also be compressed during deployment by a removable delivery sheath. Inflatable member 420 has an inflation cavity and can operatively couple to a catheter, such that the inflation cavity is in fluid communication with a lumen of a catheter. Upon reaching the treatment site, the assembly of brush 410 and inflatable member 420 can be positioned in the gutter. In the deflated configuration, inflatable member 420 can receive fluid, such as, for example, water, saline, gel, or other suitable fluid, through the catheter lumen. This fluid causes inflatable member 420 to expand from the deflated configuration. The expansion can cause inflatable member 420 and associated brush 410 to fill the gutter and engage the medical devices and/or vascular wall defining the gutter. Resulting friction created by the engagement of the bristles of brush 410 on the medical devices and/or vascular wall can further retain and stabilize inflatable member 420 in the gutter. The assembly of brush 410 and inflatable member 430 can also be releasably restrained, delivered endoluminally toward the treatment site, and then subsequently unrestrained at or near the treatment site and allowed to expand to fill the gutter where the bristles are sufficient stiff to expand inflatable member 420. Inflatable member 420 can also be filled with fluid through catheter lumen to further stabilize brush 410-inflatable member 430 assembly. The bristles of brush 410 can have openings at their distal ends for drug delivery, in which case a drug or drug mixture can be used to inflate member 420.

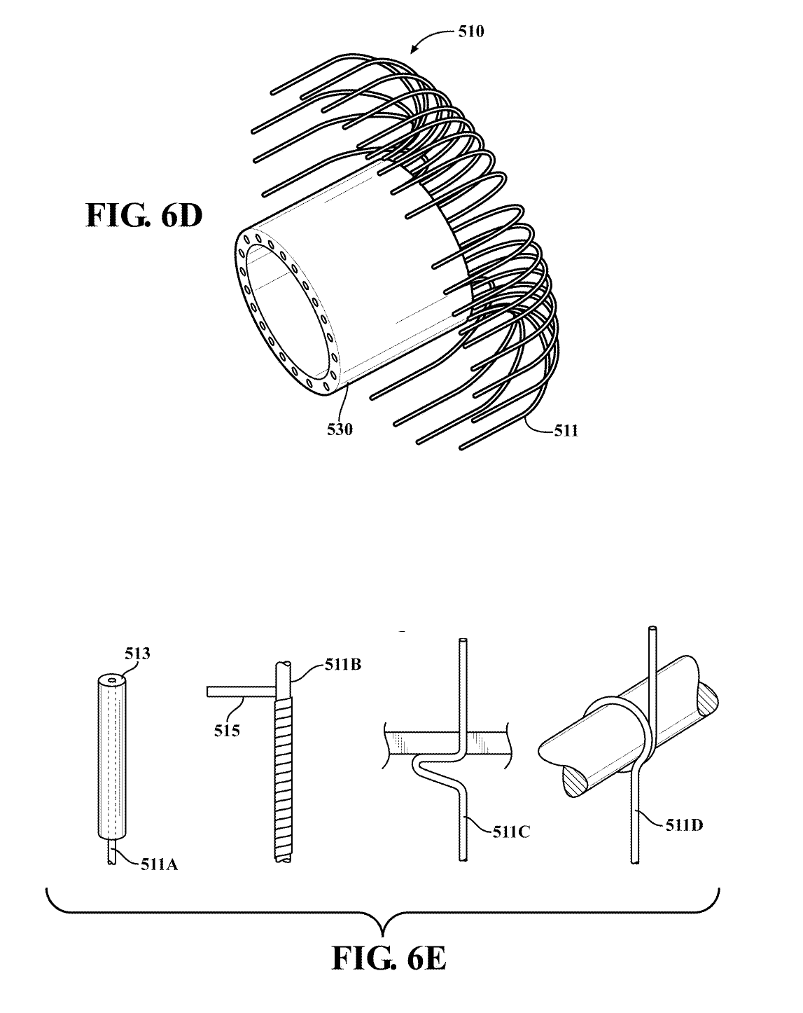

In various embodiments, for example, with reference to FIGS. 6A-6E, a leakage reduction system 500 comprises a brush 510 and a deployment mechanism 530. Brush 510 can comprise a support column 512 and a plurality of bristles 511. Support column 512 can define and/or couple to deployment mechanism 530. Bristles 511 can couple to support column 512 in any suitable orientation. For example, bristles 511 can be distributed along support column 512 in a helical orientation, in disk orientations along one or more spaced diameters of support column 512, in linear orientations along the longitudinal access of support column 512, in a uniform orientation, in a random orientation, or in any other suitable orientation. Bristles 511 can also couple to deployment mechanism 530, such that support column 512 of brush 510 is not required.

Bristles 511 can be of any suitable shape. For example, bristles 511 can be substantially straight, curve at an end, curve over substantially the entire length of bristle 511, and/or the like. Bristles 511 can be made of any suitable material, including, for example, ePTFE, metal, other types of plastics, alloys, composites, or any other suitable material. Bristles 511 can have any suitable rigidity and strength. In one embodiment, e.g., where bristles 511 are substantially soft, brush 510 employs atraumatic anchoring by increasing the number of contact points and decreasing the contact pressure. In other words, brush 510 can comprise a greater number of bristles 511 to increase the contact force exerted on a surface where brush 510 is installed and/or the surface area of the leakage reduction system. In another embodiment, e.g., where bristles 511 are substantially rigid and strong, brush 510 can have fewer bristles 511.

Bristles 511 can couple to support column 512. In one embodiment, bristle 511 comprises a first end and a second end. The first end of bristle 511 can couple bristle 511 to support column 512 in a cantilevered configuration. In this embodiment, the second end of bristles 511 protrudes outward from the column in a substantially straight or curved configuration. In another embodiment, bristles 511 comprise first and second ends. In this embodiment, both the first end and the second end of bristle 511 attach to support column 512. As such, a curved portion of the length of bristle 511 protrudes from support column 512.

In various embodiments, for example with reference to FIG. 6E, bristle 511 can be configured with enhancements. In one embodiment, bristle 511A can be configured to swell. For instance, bristle 511A can be covered with a hydrogel 513. Where bristles 511A are installed to obstruct fluid flow in a gutter, the bristles can swell to provide more effective occlusion of the gutter. In other embodiments, bristle 511B can be wrapped 515 in a fluoropolymer such as ePTFE. The wrap can provide, strength, durability, wear resistance, insertability, biocompatibility, and increased surface area, among other advantages. In these embodiments, the fluoropolymer can protect and prevent bristles 511B from becoming damaged or contaminated prior to and during deployment to a treatment region. Similarly, the fluoropolymer can protect the surrounding vasculature. The fluoropolymer coating can also act as a lubricant, for example, when brush 520 is installed within a gutter, such that bristles contact at least one of a medical device and a vascular wall. The fluoropolymer coating may also be configured with an engineered microstructure which could facilitate cellular ingrowth, which assists in mitigation of migration. Similarly, porous microstructure can be an ideal place to imbibe a particular therapeutic agent.

In another embodiment, bristles 511C can include a depth stop. The depth stop can be configured to selectively set the length of bristle that penetrates a vessel wall or protrudes from a brush and support column. In yet another embodiment, bristles 511C can be configured with “S” bends. The “S” bends can also limit how far the bristles penetrate surrounding tissues or other endovascular devices. The “S” bends can also limit how far the bristles protrude from the support column, allowing the size and shape of leakage reduction system 500 to be adjustable. In various embodiments, bristle 511D can be formed or wrapped in a way to provide a guide wire pathway for receiving a guidewire therethrough. When installed in a gutter, wrapped bristles can provide a brush with greater surface area to facilitate tissue in-growth and coagulation and/or to provide an increased surface area. Moreover, these enhancements can allow the device to be delivered over a guide wire and increase the effectiveness and life of leakage reduction system 500.

In various embodiments, brush 510 can be configured to connect to a guidewire or catheter of a medical device delivery system. Brush 510 can be collapsible or can be constrained by a sheath as brush 510 deploys endoluminally via the catheter. Upon reaching the treatment site, brush 510 can be installed in the gutter. Bristles 511 can contact the medical devices and/or vascular wall defining the gutter. The contact by bristles 511 can stabilize and retain brush 510 in the gutter. When installed, brush 510 can obstruct the gutter causing more blood and/or bodily fluid to flow through the lumens defined by the medical devices. Depending on the density of bristles 511 on brush 510 some blood and/or bodily fluid can be allowed to leak into or through the gutter.

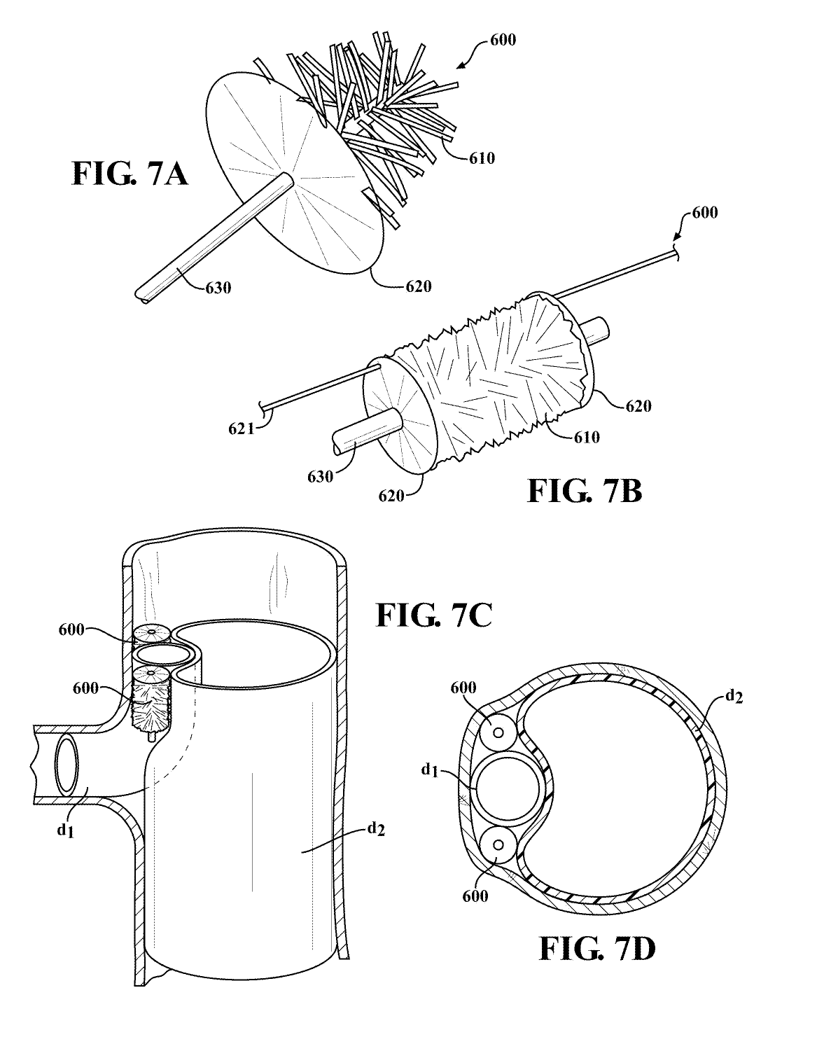

In various embodiments, for example, with reference to FIGS. 7A, 7C, 7D, leakage reduction 600 comprises a brush 610, an occluder panel 620 and a deployment mechanism 630. Brush 610 can couple to occluder panel 620. Brush 610 can also couple to or define deployment mechanism 630. Moreover, brush 610 can be configured in any suitable fashion as discussed above.

Occluder panel 620 can be any suitable structure to restrict fluid flow. Occluder panel 620 can be configured to operatively couple at any point on the proximal or distal end of brush 610. In various embodiments, one or more occluder panels 620 can couple to each of the proximal end and distal end of brush 610. Occluder panel 620 can couple to one of more bristles and provide a pathway for receiving a guide wire 621 therethrough. Occluder panel 620 can laminate a plurality of bristles, which can provide additional strength and rigidity to occluder panel 620. Where occluder panel 620 couples to the support column of brush 610, occluder panel 620 can comprise a frame and/or supports.

In various embodiments, brush 610 can couple to a guidewire and/or catheter of a medical device delivery system. Brush 610 can be compressed or restrained and deployed through a medical device delivery system to a treatment region. Upon reaching the treatment region, brush 610 can be expanded and installed in the gutter or oppose the intraluminal surface of a deployed stent. Brush 610 can be installed in the gutter, such that the bristles of brush 610 contact at least a portion of the medical devices and/or the vascular wall defining the gutter or deployed vascular device e.g. stent. Occluder panel 620 can further obstruct the gutter. Where brush 610 comprises a first occluder panel 620 at its proximal end and a second occluder panel 620 at its distal end, brush 610 can be placed in the gutter in a releasably restrained configuration. When the brush is released and unrestrained at or near a treatment site, the bristle can expand to stabilize the brush in the gutter and restrict fluid flow through the gutter. Occluder panels 620 can also expand to obstruct fluid flow through the gutter at both the proximal end and distal end of brush 610. Although occluder panels are shown with a generally circular shape, it should be appreciated that the occluder panels may have other alternate shapes or peripheries. The occluder panel on one side of the device may, for example, have a different size or shape than the occluder panel on the other side of the device. Shapes may include roughly triangular, oval, elliptical, trapezoidal etc,

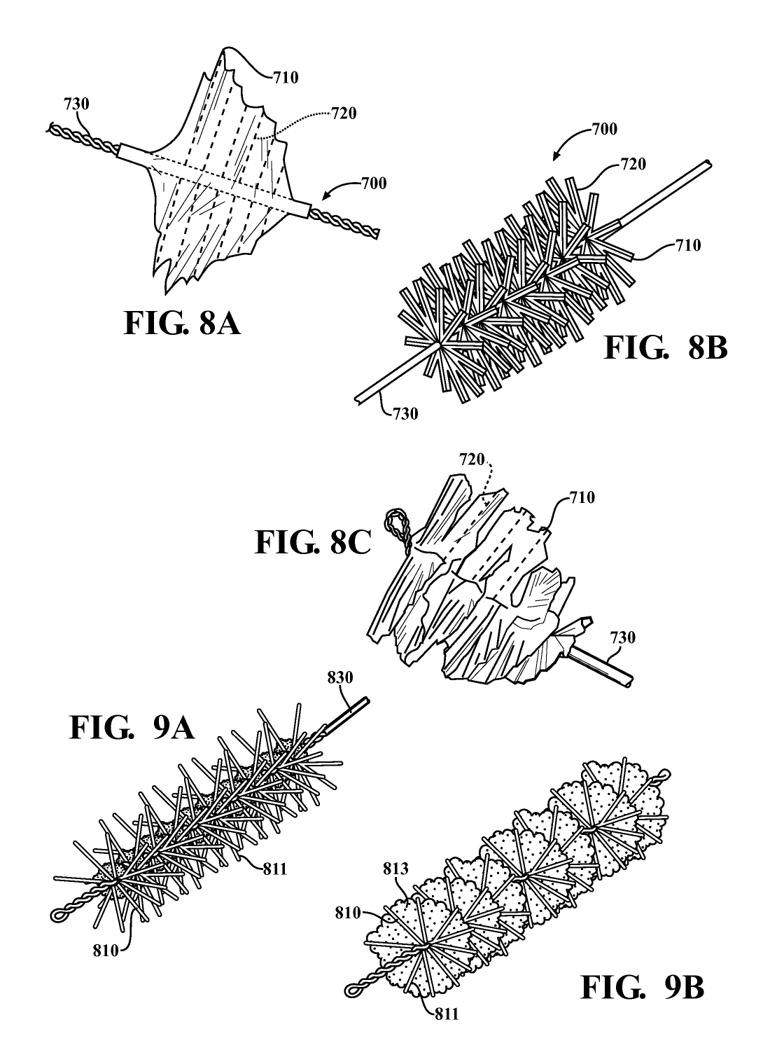

In various embodiments, for example, with reference to FIGS. 8A-8G, leakage reduction system 700 can comprise a brush 710, laminated bristles 720, and a deployment mechanism 730. Laminated bristled 720 can be laminated or covered in any suitable fashion. For example, each bristle can be laminated individually, several bristles can be laminated in a single sheet, all of the bristles can be laminated in a helical orientation, all of the bristles can be laminated together, or any other suitable lamination orientation.

Leakage reduction system 700 can deploy endoluminally through a medical device delivery system to a treatment region in a restrained or compressed configuration. In one embodiment, leakage reduction system 700 can expand and then be installed in the gutter. The laminated bristles 720 can contact the medical devices and vascular walls defining the gutter to stabilize leakage reduction system 700. The lamination coupled to the bristles can obstruct the gutter and substantially reduce the leakage through the gutter around the medical devices. Leakage reduction system 700 can also be installed in the gutter in a releasably restrained or compressed configuration and then allowed to expand, to reduce the leakage at the gutter.

In various embodiments, for example, with reference to FIGS. 9A and 9B, leakage reduction system 800 comprises a brush 810, and a deployment mechanism 830. Brush 810 can further comprise a support column and bristles. The support column can define a channel having one or more nozzles coupling the channel to an outer surface of the support column. In other embodiments, for example as illustrated in FIG. 9B, brush 810 can be configured to receive a substance 813 from a medical device delivery system though the channel defined by the support column. The substance can seep out between the bristles 811. The substance can be any suitable space filler, such as, liquid embolic space filler foam. Upon installation of the brush in the gutter region the substance can seep between the bristles to fill the voids between the bristles and substantially reduce the leakage through the gutter.

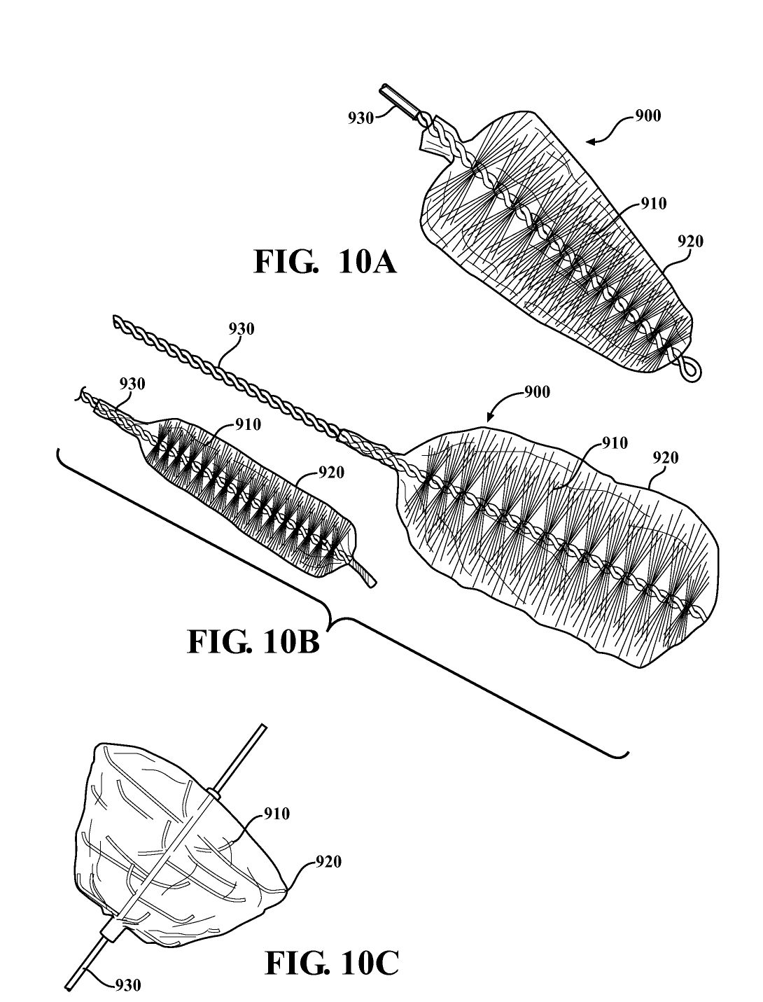

In various embodiments, for example, with reference to FIGS. 10A-10C, leakage system 900 comprises a brush 910, a bag 920, and a deployment mechanism 930. Brush 910 can install with bag 920 such that bag 920 covers at least a portion of the bristles of brush 910. Bag 920 can be of any suitable size and shape to cover brush 910. In one embodiment, bag 920 is made of a fluoropolymer such as ePTFE. However, bag 920 can be made of any suitable biocompatible material.

In other embodiments, bag 920 can be formed from a fluoropolymer having a plurality of pores of a predetermined average size to control speed of deployment of the bag. Larger pores in bag 920, for example, allow for greater fluid flows through the bag, which can allow for a lower force to expand the bag. This can be particularly useful, for example, where the bristles of brush 910 are made of a soft and flexibly material, such as, ePTFE, and may not have sufficient rigidity to expand bag 920 when bag 920 is subjected to a fluid flow. The increased fluid flow can also assist with the deployment of bag 920 by pulling the bag from a compressed configuration to an expanded configuration. Where the larger pores are not sufficient to allow for proper deployment, bag 920 can be hydrophilic. Where bag 920 is hydrophilic, the increased fluid flow through created by the hydrophilic properties can further expand bag 920. In a configuration where bag 920 is perforated, bristles from indwelling brush 910 can extend through perforations and engage the tissue and/or adjacent endovascular devices. Such engagement of bristles can minimize migration of the device.

The assembly of brush 910 and bag 920 can be of any suitable size and shape for substantially reducing the leakage through a gutter. Upon deployment through a medical device delivery system to a treatment region installation of the assembly of brush 910 and bag 920 can be expanded and installed in the gutter or installed within the gutter in a constrained configuration and allowed to expand. If the assembly of brush 910 and bag 920 tends to be a difficult to open once placed in the gutter, the assembly can be expanded prior to installation in the gutter.

In various embodiments, the leakage reduction system can be used independently to obstruct flow through a vessel; or to occlude anatomical features such as left atrial appendage; or to obstruct flow though a cardiac defect.

Thus, the leakage reduction system described herein provides a mechanism to obstruct and substantially reduce the leakage through a gutter.

The support structures, coatings and secondary structures, described above, are highly bio-compatible. As used herein, a “biocompatible material” is a material suited for and meeting the purpose and requirements of a medical device, used for either long or short term implants or for non-implantable applications. Long term implants are defined as items implanted for more than 30 days. These support structures, coatings, and secondary structures can be formed of a fluoropolymer, such as ePTFE. Alternatively, or in combination with a fluoropolymer, the support structures, coatings, and secondary structures can be formed of biocompatible materials, such as polymers, which can include fillers such as metals, carbon fibers, Dacron, glass fibers or ceramics. Such polymers can include olefin polymers, polyethylene, polypropylene, polyvinyl chloride, polytetrafluoroethylene which is not expanded, fluorinated ethylene propylene 45 copolymer, polyvinyl acetate, polystyrene, poly(ethylene terephthalate), naphthalene dicarboxylate derivatives, such as polyethylene naphthalate, polybutylene naphthalate, polytrimethylene naphthalate and trimethylenediol naphthalate, polyurethane, polyurea, silicone rubbers, polyamides, polycarbonates, polyaldehydes, natural rubbers, polyester copolymers, styrene-butadiene copolymers, polyethers, such as fully or partially halogenated polyethers, copolymers, and combinations thereof. Also, polyesters, including polyethylene terephthalate (PET) polyesters, polypropylenes, polyethylenes, polyurethanes, polyolefins, polyvinyls, polymethylacetates, polyamides, naphthalane dicarboxylene derivatives, and natural silk can be included in support structures, coatings and secondary structures.

These support structures, coatings and secondary structures can be utilized with bio-active agents. Bio-active agents can be coated onto a portion or the entirety of the support structures, coatings and secondary structures for controlled release of the agents once the support structures, coatings and secondary structures is implanted. The bio-active agents can include, but are not limited to, vasoconstrictors, and thrombogenic agents, such as thrombin. Bio-active agents can also include, for example, vasodilators, anti-coagulants, such as, for example, warfarin and heparin. Other bio-active agents can also include, but are not limited to agents such as, for example, anti-proliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP) IIb/IIIa inhibitors and vitronectin receptor antagonists; anti-proliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); anti-proliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetominophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blockers; nitric oxide donors; anti-sense oligionucleotides and combinations thereof; cell cycle inhibitors, mTOR inhibitors, and growth factor receptor signal transduction kinase inhibitors; retenoids; cyclin/CDK inhibitors; HMG co-enzyme reductase inhibitors (statins); and protease inhibitors.

As used herein, the term “bio-resorbable” includes a suitable bio-compatible material, mixture of materials or partial components of materials being degraded into other generally non-toxic materials by an agent present in biological tissue (i.e., being bio-degradable via a suitable mechanism, such as, for example, hydrolysis) or being removed by cellular activity (i.e., bioresorption, bioabsorption, or bioresorbable), by bulk or surface degradation (i.e., bioerosion such as, for example, by utilizing a water insoluble polymer that is soluble in water upon contact with biological tissue or fluid), or a combination of one or more of the bio-degradable, bio-erodable, or bio-resorbable material noted above. Potential materials for the stent described herein include, for example, biodegradable polymers such as polylactic acid, i.e., PLA, polyglycolic acid, i.e., PGA, polydioxanone, i.e., PDS, polyhydroxybutyrate, i.e., PHB, polyhydroxyvalerate, i.e., PHV and copolymers or a combination of PHB and PHV (available commercially as Biopol®, polycaprolactone (available as Capronor®), polyanhydrides (aliphatic polyanhydrides in the back bone or side chains or aromatic polyanhydrides with benzene in the side chain), polyorthoesters, polyaminoacids (e.g., poly-L-lysine, polyglutamic acid), pseudo-polyaminoacids (e.g., with back bone of polyaminoacids altered), polycyanocrylates, or polyphosphazenes; as well as bioresorbable metals or metal alloys.

Finally, the present disclosure has been described above with reference to various embodiments. It should be appreciated that the various embodiments shown and described herein are illustrative of the present disclosure and its best mode and are not intended to limit in any way the scope of the present disclosure. Those skilled in the art having read this disclosure will recognize that changes and modifications can be made to the various embodiments without departing from the scope of the present disclosure. For example, various aspects and embodiments of the present disclosure can be used to provide other methods of treatment, such as drug eluting stents, and/or for imaging purposes. Although certain preferred aspects of the present disclosure are described herein in terms of embodiments, such aspects of the present disclosure can be achieved through any number of suitable means now known or hereafter devised. Accordingly, these and other changes or modifications are intended to be included within the scope of the present disclosure.

What is claimed is:

1. An implantable device assembly comprising:
a gutter filler configured to obstruct a gutter defined between at least a portion of a vascular wall and at least two or more medical devices in a treatment region, and to reduce an unwanted flow around the at least two or more medical devices;
an elongated delivery device for supporting the gutter filler for endoluminal delivery of the gutter filler to the treatment region; and
a deployment mechanism that removably interconnects the gutter filler and the elongated delivery device allowing separation of the elongated delivery device from the gutter filler and removal of the elongated delivery device from the treatment region after installation of the gutter filler at the treatment region,
wherein the gutter filler includes a brush having a plurality of bristles and a bag covering a substantial portion of the brush.

2. The implantable device of claim 1, wherein the gutter filler comprises a frame that supports the bag.

3. The implantable device of claim 1, wherein the bag surrounds at least one of foam, a gel, a multi-part substance, and beads.

4. The implantable device of claim 1 further comprising an anchor configured to couple to a vascular wall and stabilize the gutter filler.

5. The implantable device of claim 1, wherein the bag is inflatable.

6. The implantable device of claim 5, wherein the bristles include openings for delivery of fluid into the bag to cause inflation of the bag.

7. The implantable device of claim 5, wherein the bag has a textured surface.

8. The implantable device of claim 7, wherein the textured surface is at least one of a raised portion of bag material and a protrusion of a structure within the bag.

9. The implantable device of claim 1, wherein the bag comprises ePTFE.

10. The implantable device of claim 1, wherein the bristles of the brush comprise at least one of polymer and metal alloy.

11. An implantable device comprising:
a catheter; and
a gutter filler releasably coupled to a leading end of the catheter and configured to obstruct a gutter defined between at least a portion of a vascular wall and at least one medical device in a treatment region and thereby reduce an unwanted flow around the at least one medical device,
wherein the gutter filler includes a brush having a plurality of bristles and a bag covering a substantial portion of the brush.

12. The implantable device of claim 11, wherein the gutter filler comprises a frame and the bag is coupled to the frame.

13. The implantable device of claim 12, wherein the bag is filled with at least one of foam, a gel, a multi-part substance, and beads.

14. The implantable device of claim 11, wherein the bag is inflatable.

15. The implantable device of claim 14, wherein the bristles include openings for delivery of fluid into the bag to cause inflation of the bag.

16. The implantable device of claim 14, wherein the bag has a textured surface.

17. The implantable device of claim 16, wherein the textured surface is at least one of a raised portion of bag material and a protrusion of a structure within the bag.

18. The implantable device of claim 17, wherein the bag comprises ePTFE.

19. The implantable device of claim 11, wherein the bristles of the brush comprise at least one of polymer and metal alloy.

20. The implantable device of claim 11 further comprising an anchor configured to couple to a vascular wall and stabilize the gutter filler.
